(19) United States
(12) Patent Application Publication
(10) Pub. No.: US 2005/0191644 A1
(43) Pub. Date: Sep. 1, 2005

(54) Methods for detecting biopolymers; biochips; methods for immobilizing antibodies; and substrates to which antibodies are immobilized

(75) Inventors: Nobuo Shimamoto, Mishima (JP); Motoki Susa, Sunto (JP); Kazuhisa Fukushima, Musashino-shi (JP)

Correspondence Address: SEED INTELLECTUAL PROPERTY LAW GROUP PLLC, 701 FIFTH AVE, SUITE 6300, SEATTLE, WA, 98104-7092, US

(73) Assignees: Research Organization of Information and Systems, President, Minato-ku (JP); Yokogawa Electric Corporation, Musashino-shi (JP)

(21) Appl. No.: 10/960,849

(22) Filed: Oct. 6, 2004

(30) Foreign Application Priority Data
Dec. 16, 2003 (JP) 2003-417494
Oct. 6, 2003 (JP) 2003-347072

(51) Int. Cl. C12Q 1/68; G01N 33/53; C12M 1/34; H01L 21/00

(52) U.S. Cl. 435/6; 435/7.1; 435/287.2; 438/1

(57) ABSTRACT

The present invention relates to biopolymer detection methods based on antigen-antibody interactions, wherein the methods show improved S/N ratios, improved detection sensitivities, and reduced detection times. The present invention also relates to application of these methods to biochips. The invention further relates to antibody fixation methods wherein antibody molecules are immobilized to amide group-containing gels, or amide group-containing gels on insoluble substances. Using such gels prevents the nonspecific adsorption of antibody-binding molecules. By embedding these antibody-binding molecules in such gels, their antibody-binding activity is prevented from deteriorating. The methods for detecting biopolymers by trapping target biopolymers to the substrate side, comprise the steps of: 1) placing target biopolymers, with probe biopolymers and beads that are identified by antibodies or address probe peptides or biopolymer address linkers attached to their surface, in a solution; 2) hybridizing the target biopolymers with the probe biopolymers; and 3) identifying the address linkers bound to a substrate by the address probe peptide or biopolymers or polyclonal antibody molecules through antigen-antibody interactions. The methods for immobilizing antibodies comprise the steps of: 1) applying an amide group-containing gel embedded with antibody-binding molecules on a substrate of an insoluble substance in two or three dimensions; and 2) attaching the base of the antibody molecules to antibody-binding molecules.

### CROSS-REFERENCE TO RELATED APPLICATION

This application claims the benefit of Japanese Provisional Patent Application Nos. JP 2003-417494, filed Dec. 16, 2003 and JP 2003-347072, filed Oct. 6, 2003, which provisional applications are incorporated herein by reference in their entireties.

### BACKGROUND OF THE INVENTION

1. Field of the Invention

The present invention describes methods for detecting biopolymers such as deoxyribonucleic acids (DNAs), ribonucleic acids (RNAs), and proteins; and biochips using these methods. Furthermore, the present invention relates to methods for immobilizing antibodies, and to substrates to which the antibodies are immobilized.

2. Description of the Related Art

Techniques for detecting biopolymers (hereinafter taking DNA as an example) by using microarrays are well known, for example that described in Unexamined Published Japanese Patent Application No. (JP-A) 2000-131237. These type of DNA microarrays are usually formed and used to detect DNAs as follows:

Arrays of DNA probes comprising sequences complementary to the target mRNAs (or cDNAs) are spotted and then immobilized to a glass (or plastic) substrate. The substrate is exposed to a set of fluorescence-labeled target mRNAs (or cDNAs) in solution. At this point, complementary probes and target mRNAs (or cDNAs) hybridize and bind to form a complex. Targets whose sequences are not complementary to a probe will not hybridize. When hybridization has proceeded adequately, the substrate surface is washed with a buffer solution, and any unbound target molecules are washed away.

The presence or absence of target mRNAs (or cDNAs), and their quantities, can be optically determined from fluorescent intensities at the spots where the hybrids locate. Such optical measurement methods are well established, and one such example is described in detail in JP-A 2000-235035.

The use of conventional DNA microarray protocols, such as the one described above does not provide satisfactory results. Each step in each protocol poses numerous problems, including data accuracy, reproducibility, repeatability, and sensitivity, which hampers the standardization of experimental data. In addition to difficulties in target cDNA selection, i.e., difficulties with disease contents sequence selection, these problems prevented DNA microarray techniques from prevailing into clinical applications.

In solving the above-mentioned problems it is essential to improve S/N ratio, detection sensitivity, detection time, data accuracy, and reproducibility.

### BRIEF SUMMARY OF THE INVENTION

The present invention is intended to solve the aforementioned problems. Specifically, an objective of the present invention is to provide methods for detecting biopolymers based on antigen-antibody interactions and bead techniques to improve S/N ratio and detection sensitivity, as well as to reduce detection time. Another objective of the present invention is to provide biochips to be used in the invented methods.

### BRIEF DESCRIPTION OF THE DRAWINGS

FIG. 1 is a schematic view illustrating the principle of an exemplary method for detecting biopolymers in the present invention.

FIG. 2 is a second schematic view illustrating the principle of the exemplary method for detecting biopolymers in the present invention.

FIG. 3 is a third schematic view illustrating the principle of the exemplary method for detecting biopolymers in the present invention.

FIG. 4 is a schematic view illustrating the principle of a method for immobilizing the antibody molecules of another example of the present invention.

FIG. 5 is a schematic view illustrating how antibody molecules are immobilized to a polyacrylamide gel substrate.

### DETAILED DESCRIPTION OF THE INVENTION

In the present invention, the advantages of beads and DNA microarrays are combined. The beads provide a greater surface area per volume than a flat plate, and accordingly allow more probe DNAs to be bound. Furthermore, there is an increased frequency of collision between probe DNAs and target molecules due to the increased mobility of beads in a solution compared to a flat plate. Consequently, target DNAs in the solution can be trapped with an improved sensitivity.

As a drawback, identifying the individual beads to which probe DNAs are bound is required. Various techniques, such as the use of colored beads and a two-color light source, have been tried to solve this problem. However, the number of beads that can be successfully identified is still small, and the equipment becomes more complicated, more expensive, larger, and more difficult to handle. The present invention provides the perfect solution to these problems by utilizing the antigen-antibody interaction that takes place between the peptide antigen fixed on beads and the antibodies immobilized in an array, or vice versa.

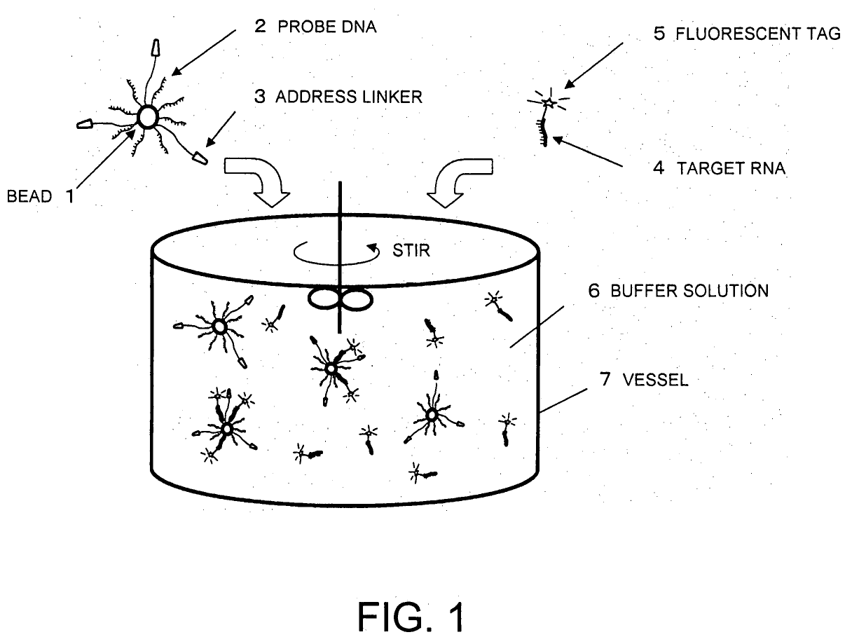

The present invention is hereafter described in detail with reference to FIGS. 1 to 3. FIGS. 1 to 3 are schematic views illustrating the principle of the methods for detecting biopolymers of the present invention. Please note that herein the explanations refer to DNA biopolymers.

As shown in FIG. 1, probe DNA (2) is immobilized to the surface of bead (1). The beads can be, for example, magnetic, metallic, and/or plastic. Address linker (3) is attached to the surface of bead (1) to enable recognition of the bead's identification information (ID). Address linker (3) can be an antigen or a (monoclonal or polyclonal) antibody. A fluorescent tag (5) is used to label target (4), which can be an RNA, cDNA, or protein (hereinafter these are represented by reference to “RNA”).

Bead (1) and target RNA (4), prepared as described above, are mixed in buffer solution (6) in vessel (7), by physical or electrical means as necessary. As a result, target RNA (4) binds to probe DNA (2), which is immobilized to the surface of bead (1) via complementary base pairing.

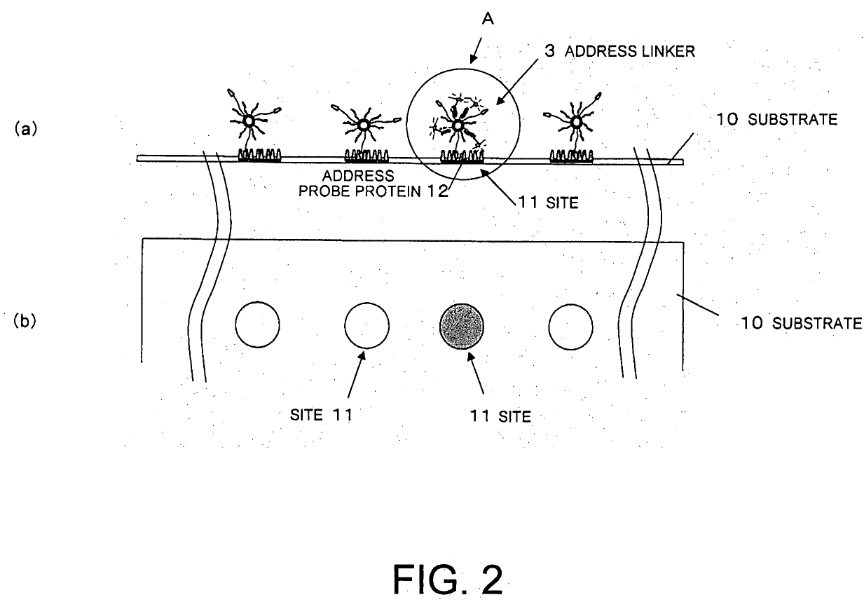

The beads carrying the aforementioned complexes are then incubated with arrays of site (11) on a substrate (10) of the biochip illustrated in FIG. 2. FIG. 2A and FIG. 2B present side and plan views of the biochip, respectively.

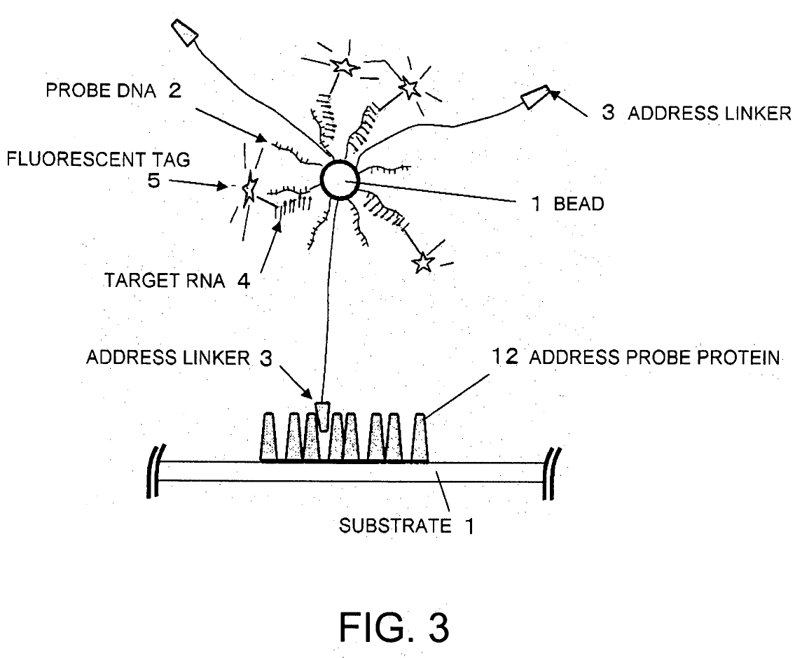

Address probe protein (12), such as an antigen or antibody, is pre-immobilized to site (11) to trap ID-recognizing address linker (3), which is immobilized on the surface of bead (1), via an antigen-antibody reaction. Please note that FIG. 3 is an enlargement of area A, which is circled in FIG. 2A.

Address linker (3) binds to address probe protein (12) via an antigen-antibody interaction. Fluorescent tag (5) can be used to identify which address probe protein (12) in probe site (11) was bound to bead (1). The fluorescent labels can be easily detected using a fluorescence reader (not shown).

Thus, it is possible to effectively determine the presence and amount of target RNA 4.

For instance, an address probe peptide or biopolymer other than a polyclonal antibody can be used as an address linker. In such cases, one of an address probe peptide, biopolymer, or polyclonal antibody, each of which has an antigen-antibody relationship with a specific address linker, is immobilized to the substrate.

The present invention comprises the following advantages:
(1) A large amount of a probe DNA can be immobilized since beads have a comparatively large surface area to volume ratio. Consequently, trace quantities of target biopolymers in a solution can be trapped with an extremely high sensitivity.
(2) The increased amount of probe DNAs per bead can hybridize with more target DNAs, and therefore can enhance the S/N ratio.
(3) There are increased chances for probe DNAs and target DNAs to collide with each other due to the mobility of the beads. Consequently, detection time (mainly the time required for hybridization) is reduced, and hybridization between the target DNAs and probe DNAs can be extremely sensitive.

The following embodiments describe methods for immobilizing antibodies, and substrates to which the antibodies are immobilized, where these methods can be used in the aforementioned methods for detecting biopolymers, and in biochips that apply these detection methods.

As the above-mentioned example demonstrates, antibody-binding molecules specifically can bind to specific antigen molecules. These antibodies are normally used after being immobilized to an insoluble substance, such as plastic or metal (equivalent to the substrate of the above-described example). Most of these insoluble substances adsorb biopolymers non-specifically.

One method for immobilizing antibodies to insoluble substances is by using antibody-binding molecules immobilized to the insoluble substance. In such cases, the antibody-binding molecules are immobilized to the insoluble substance via covalent bonding, or via non-covalent bonding such as electrostatic interactions. This is described in, for example, JP-A 2001-147229.

Nevertheless, conventional methods present the following defects:
(1) Immunoassays, fractionation, and purification may be prevented since the insoluble substances nonspecifically adsorb the biopolymers.
(2) Molecules may lose their antibody-binding activity due to secondary (nonspecific) interactions between the insoluble substance and the antibody-binding molecules that result when they are placed in extremely close proximity with the insoluble substance for binding.

The following Example solves these problems by using an amide group-containing gel to prevent the non-specific adsorption of antibody-binding molecules, as well as to prevent deterioration of antibody-binding activity by embedding the antibody-binding molecules in to the amide group-containing gel. Thus, in the present invention, antibody molecules are immobilized to an amide group-containing gel or an amide group-containing gel on an insoluble substance.

In this Example, the present invention uses a polyacrylamide gel as the substrate material. Furthermore, the substrate is prepared in such a way that the antibody-binding molecules are embedded in the polyacrylamide gel, and the deterioration of their antibody-binding activity can be prevented through binding with the amide-group carriers in the gel.

This Example of the present invention also comprises the following attributes: Since the antibody-binding molecules are embedded in a polyacrylamide gel, their antibody-binding activity will not be degraded by binding with carriers. By introducing antibody-binding molecules into a gel, the molecules are maintained in a state close to the state in solution where they are functionally active. Furthermore, immobilization by means of embedding does not depend on an antibody-binding molecule's functional group, but rather depends primarily on the size of the holes formed by the embedded substance.

Polyacrylamide gels embedding antibody-binding molecules prepared as described above can be provided directly, or backed up by an insoluble substance. Antibody are immobilized by immersing the polyacrylamide gel, or the insoluble-substance supporting the polyacrylamide gel, in an antibody solution.

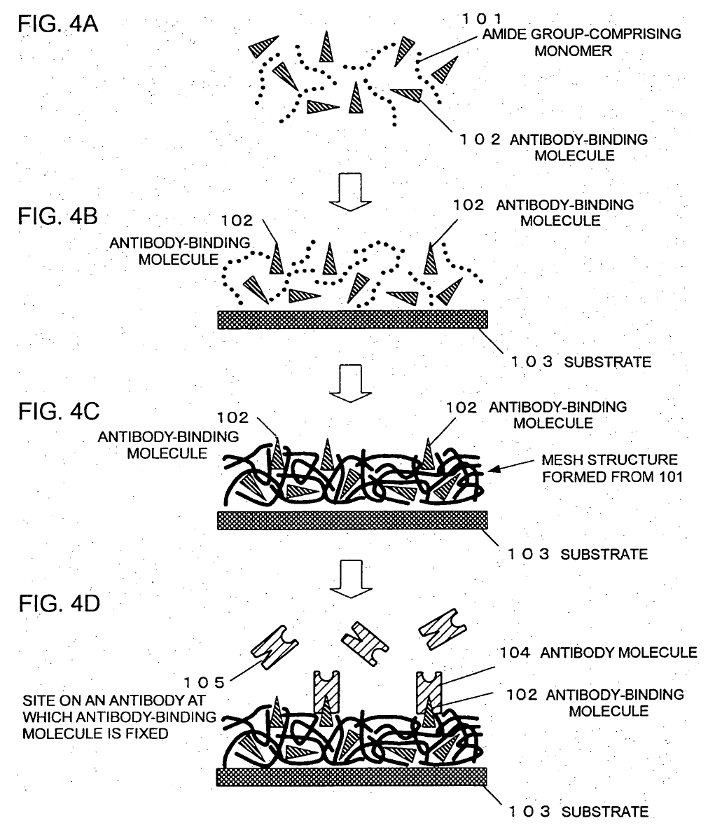

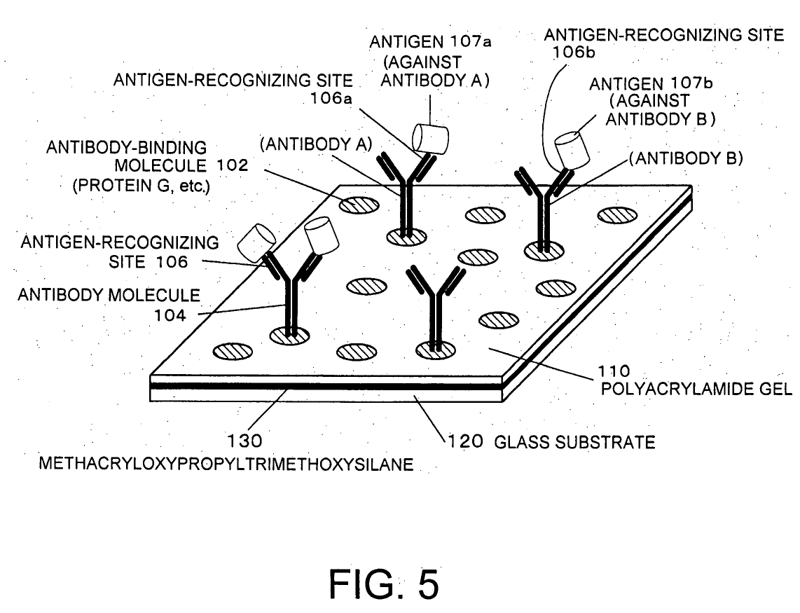

The present invention is hereinafter described in detail with reference to FIGS. 4 and 5. FIG. 4 is a schematic view illustrating the principle of a method for binding antibody molecules, and FIG. 5 is a schematic view illustrating how antibody molecules are immobilized to a polyacrylamide gel substrate.

The procedure for binding antibody molecules is described below: As illustrated in FIG. 4(A), amide group-containing monomers (101) and antibody-binding molecules (102) are mixed together. This mixture is applied to the surface of substrate (103) (an insoluble substance), as illustrated in FIG. 4(B), and then polymerization is carried out. An insoluble polymer compound, such as a metal, plastic or glass, is used as substrate (103). Through this polymerization reaction, antibody-binding molecules (102) are trapped within the mesh structure formed by the amide group-containing gel (hereinafter a polyacrylamide gel is used as an example of such gels) (see FIG. 4(C)).

When immobilizing antibodies, a solution comprising antibody molecules (104) is applied in drops, as illustrated in FIG. 4(D). Antibody molecules can be monoclonal or polyclonal antibodies. Antibody-binding molecule (102) is immobilized in a polyacrylamide gel. Antibody molecule (104) binds to this antibody-binding molecule (102) at one or more points at its site (105) specific for the binding. Antibody-binding molecule (102) is arranged such that it is physically embedded in the cage of polyacrylamide gel. Consequently, the base of antibody molecule (104) is immobilized to antibody-binding molecule (102), leaving its antigen-binding site exposed and accessible to antigen molecules.

FIG. 5 is a schematic view illustrating a substrate for immobilizing antibody molecules based on the aforementioned principles. This figure illustrates how an antibody molecule (104) is immobilized to a substrate via antibody-binding molecule (102) embedded in polyacrylamide gel (110). This figure also shows antibody-binding molecule (102) addressed by many different varieties of antibodies, such as antibodies against antigens A and B.

In this figure, substance (120) is an insoluble substance such as glass. The surface of glass substrate (120) is treated with methacryloxypropyltrimethoxysilane 130. Polyacrylamide gel (110) is applied to the surface upon polymerization, which helps to adhere polyacrylamide gel (110) to glass (120).

As illustrated in FIG. 4, antibody-binding molecule (102) (a biopolymer or a polymer compound with antibody-binding activity, such as protein G or its analogues) is embedded non-covalently in polyacrylamide gel (110). The base of antibody molecule (104) is bound to antibody-binding molecule (102), as illustrated in FIG. 5, so that its antigen-recognizing site 106 is in contact with the solution (facing up in the figure). In this manner, a corresponding antigen binds to the antigen-recognizing site 106 of antibody molecule 104. More specifically, antigen 107a against antibody A binds to antibody A's antigen-recognizing site, 106a, and antigen 107b against antibody B binds to antibody B's antigen-recognizing site, 106b.

An amide-containing substance such as polyacrylamide gel (110) significantly reduces nonspecific adsorption and thus increases the exposure of antigen-recognizing site (106) to the solution. These two advantages further improve the efficiency and specificity of the antibody-antigen interactions.

The above-mentioned example demonstrates the following effects of the present invention:
(1) Since 1) antibody binding molecules are combined with the amide-containing gel in the substrate, and 2) the antibody binding molecules trap the basal portion of the antibody, which is the reverse portion of the antibody-antigen binding portion, and 3) the antibody-antigen binding portion of the antibody is thus free to actively combine with antigens, non-specific binding has become reduced and uniform direction alignment has become improved. Accordingly, the specificity of antibody-antigen binding is improved.
(2) Consequently, an antibody-immobilized substrate useful for highly-sensitive immunoassays or for fractionation or for purification by using antigen molecules can be devised.

The preferred examples specifically described herein are intended merely to explain and illustrate the present invention, and the present invention is not limited to these examples; rather, alterations and modifications may be made without departing from the ideas and essential techniques of the invention. The following claims thus incorporate such alterations and modifications.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for detecting a biopolymer by trapping a target biopolymer to the substrate side, which comprises the steps of:
a) adding a fluorescent labeled target biopolymer and a bead, wherein a probe biopolymer and an address linker that specifies the ID of the bead are immobilized on the surface of the bead in solution (the address linker could be on antibody, peptide, or other biopolymer);
b) hybridizing the target biopolymer with the probe biopolymer; and
c) trapping the address linker through antigen-antibody interactions by using an address probe peptide, biopolymer, or antibody which is immobilized on a substrate and which is in an antigen-antibody relationship with the address linker.

2. The method of claim 1, wherein a buffer solution is added in with the target biopolymer and the bead; and the solution is stirred by physical or electrical means.

3. The method of claim 1, wherein the bead is a magnetic, metallic, or plastic bead.

4. The method of claim 1, wherein the target biopolymer is an RNA, a cDNA, or a protein.

5. A biochip wherein 1) an address probe peptide, biopolymer, or antibody, each of which can trap an address linker through an antigen-antibody reaction, is immobilized on to a substrate, 2) wherein the address linker is for ID recognition of a bead, and is an antibody, address probe peptide, or biopolymer, and 3) wherein the address linker is immobilized on the surface of the bead together with a probe biopolymer that binds with a target biopolymer by hybridization.

6. A method for immobilizing an antibody, which comprises the steps of 1) immobilizing an amide group-containing gel, in which an antibody-binding molecule is embedded, on to a substrate made of an insoluble substance; and 2)
binding the base of the antibody molecule to the antibody-binding molecule.

7. The method of claim 6, wherein the antibody-binding molecule is embedded non-covalently in the amide-containing gel, and where it is a biopolymer or polymer compound comprising antibody-binding activity, such as protein G or a protein of that family.

8. The method of claim 6, wherein an insoluble polymer compound such as a metal, plastic, or glass is used as the insoluble substance.

9. The method of claim 6, wherein the amide group-containing gel is a polyacrylamide gel.

10. The method of claim 6, wherein the antibody-binding molecule binds to the antibody molecule in one or more sites on the antibody molecule.

11. A substrate to which an antibody is immobilized by binding with an antibody-binding molecule, where the antibody-binding molecule is embedded in an amide group-containing gel on an insoluble substance via non-covalent binding.

12. The substrate of claim 11, wherein the antibody-binding molecule is embedded non-covalently in the amide group, where it is a biopolymer or polymer compound comprising antibody-binding activity, such as protein G or a protein of that family.

13. The substrate of claim 11, wherein an insoluble polymer compound such as a metal, plastic, or glass is used as the insoluble substance.

14. The substrate of claim 11, wherein the amide group-containing gel is a polyacrylamide gel.

15. The substrate of claim 11, wherein the surface of the insoluble substance is treated with methacryloxypropyltrimethoxysilane and the amide group-containing gel is applied to the surface upon polymerization.

16. The method of claim 1, wherein the address linker is immobilized to the substrate by the method of claim 6.

17. The biochip of claim 5, wherein the substrate of claim 11 is used for immobilizing the address probe peptide, biopolymer, or polyclonal antibody.
